(19) United States
(12) Patent Application Publication
(10) Pub. No.: US 2014/0005153 A1
(43) Pub. Date: Jan. 2, 2014

(54) Off-Note Masking

(71) Applicants: Kimberley Gray, Loveland, OH (US); Yili Wang, Mason, OH (US); Scott Wayne Keller, Cincinnati, OH (US)

(72) Inventors: Kimberley Gray, Loveland, OH (US); Yili Wang, Mason, OH (US); Scott Wayne Keller, Cincinnati, OH (US)

(73) Assignee: GIVAUDAN S. A., Vernier (CH)

(21) Appl. No.: 14/001,989

(22) PCT Filed: Mar. 14, 2012
(86) PCT No.: PCT/EP2012/054426

§ 371 Date: Aug. 28, 2013

Related U.S. Application Data
(60) Provisional application No. 61/452,456, filed on Mar. 14, 2011.

(51) Int. Cl. A23L 1/22; A61K 47/12; A61K 47/22

(52) U.S. Cl. CPC A23L 1/22075; A61K 47/22; A61K 47/12
USPC 514/171; 546/340; 426/442; 426/537

(57) ABSTRACT

A method of masking an off-note in an orally-receivable or -ingestible composition, including the addition thereto of an off-note masking quantity of 1-(2-hydroxyphenyl)-3-(pyridin-4-yl)propane-1-one or a salt thereof. The effect may be further enhanced by the addition of Compound 2 and/or 2-[(3-ethyl-4,8-dimethyl-2-oxo-2H-chromen-7-yl)oxy]propanoic acid, or salts thereof.

This disclosure relates to the masking of off-notes or off-tastes and to compounds and compositions for the masking of such off-notes or off-tastes.

Many orally-receivable or -ingestible compositions, by which is meant anything taken orally, either for ingestion or for spitting out, have off-tastes. Such off-tastes can be inherent to the composition itself, or they can be a result of a particular additive. Examples of compositions having an off-taste include coffee, soy milk and soy yoghurt. In the case of additives, particular examples are artificial sweeteners, which have been used in such compositions as a replacement for sugar and corn syrup for dietary or health reasons. These compounds, examples of which include saccharin, acesulfame-K and aspartame, are considerably sweeter than sugar, and so can be used in small amounts, replacing a considerably greater quantity of sugar. However, it has been noted that, although the predominant taste sensation of these compounds is sweetness, they frequently additionally provide an undesirable off-taste, often of a bitter or metallic quality, something absent from sugar and corn syrup. Such off-tastes limit the acceptance of compositions including such compounds, even though they are much healthier.

It has been proposed to add compounds that mask such off-tastes. A typical example of such a compound may be found in United States published application No. 2010/227039.

It has now been found that another compound has excellent off-note masking qualities for a wide range of off-tastes or off-notes. There is therefore provided a method of masking an off-note in an orally-receivable or -ingestible composition, comprising the addition thereto of an off-note masking quantity of 1-(2-hydroxyphenyl)-3-(pyridin-4-yl)propane-1-one or a salt thereof.

There is also provided an orally-receivable or -ingestible composition comprising an off-note-masking proportion of 1-(2-hydroxyphenyl)-3-(pyridin-4-yl)propane-1-one or a salt thereof 1-(2-hydroxyphenyl)-3-(pyridin-4-yl)propane-1-one (hereinafter referred to as “Compound 1”) is disclosed in United States published application No. 2010/272656. In this publication, the compound is described as an effective sweetness enhancer. It is a surprising feature of this disclosure that the same compound can deliver an off-note- or off-taste-masking effect at a concentration far below the concentration at which it is perceived as sweet. The minimum level at which the sweetness enhancement effect of Compound 1 can be detected is 35 ppm, but the off-note- or off-taste-masking effect of the masker can be as low as 2.5 ppm. A practical maximum for off-note masking is 10 ppm.

In a further embodiment, the off-note-masking effect of Compound 1 may be enhanced yet further by the combination of certain other compounds with Compound 1. The disclosure therefore also provides a method of masking an off-note in an orally-receivable or -ingestible composition, comprising the addition thereto of an off-note masking quantity of a masking composition consisting of 1-(2-hydroxyphenyl)-3-(pyridin-4-yl)propane-1-one or a salt thereof and at least one of 4-[2,2,3-trimethyl-cyclopentyl]-butanoic acid or 2-[(3-ethyl-4,8-dimethyl-2-oxo-2H-chromen-7-yl)oxy]propanoic acid, or salts thereof.

4-[2,2,3-trimethyl-cyclopentyl]-butanoic acid and 2-[(3-ethyl-4,8-dimethyl-2-oxo-2H-chromen-7-yl)oxy]propanoic acid (hereinafter respectively “Compound 2” and “Compound 3”) are disclosed in United States Patent Publications US 2010/0284944 and US 2010/0227039 respectively.

Suitable salts of the Compounds 1, 2 or 3 may include but are not limited to at least one of K⁺, NH₄⁺, Na⁺, Ca²⁺, Mg²⁺, Al³⁺, Zn²⁺, hydrochloride or hydrobromide salt. Typical ratios of the compounds in masking compositions are now given. These are given by way of general indication only, and are not intended to be in any way limiting. A skilled person can create desirable effects by working outside these proportions.

Compound 1:Compound 2 ratio 1:40 up to 40:1, particularly 1:20 up to 20:1, more particularly 1:7 up to 6:1.

Compound 1:Compound 3 ratio 1:40 up to 40:1, particularly 1:20 up to 20:1, more particularly 1:7 up to 3:1.

Compound 1 and the masking composition may be used in all variety of orally-receivable and ingestible products. Non-limiting examples of such products include:

Consumable products, including, but not limited to all food products, food additives, nutraceuticals, pharmaceuticals and any product placed in the mouth including chewing gum, oral care products, and oral hygiene products including but not limited to, cereal products, rice products, tapioca products, sago products, baker's products, biscuit products, pastry products, bread products, confectionery products, dessert products, gums, chewing gums, mouthwash, dental floss, flavored or flavor-coated straws, flavor or flavor-coated food/beverage containers, chocolates, ices, honey products, treacle products, yeast products, baking-powder, salt and spice products, savoury products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts, cheese products, butter and butter substitute products, milk substitute products, soy products, edible oils and fat products, medicaments, beverages, carbonated beverages, alcoholic drinks such as beers, wines and spirits, non-alcoholic drinks such as soft drinks, mineral and aerated waters, fruit drinks, fruit juices, coffee, artificial coffee, tea, cocoa, including forms requiring reconstitution including, without limitation, beverage powder, milk based beverage powder, sugar-free beverage powder, beverage syrup, beverage concentrate, instant coffee, instant tea, instant cocoa, and coffee whitener, food extracts, plant extracts, meat extracts, condiments, gelatins, pharmaceutical and non-pharmaceutical gums, tablets, lozenges, drops, emulsions, elixirs, syrups and other preparations for making beverages, and combinations thereof.

Oral care products, including, but not limited to, any composition applied to the oral cavity for the purposes of cleaning, freshening, healing, deodorising the cavity or any part thereof, may include, but are not limited to, toothpastes, tooth gels, tooth powders, tooth whitening products, mouthwashes, lozenges, dental floss, toothpicks, anti-plaque and anti-gingivitis compositions, throat lozenges, throat drops, inflammatory compositions, compositions for treatment of nasal symptoms, cold symptoms and upper gastrointestinal tract distress, compositions for cold relief, for alleviating discomfort of hot flash, and gargle compositions.

Typical, non-limiting examples of Compound 1 are:

in a beverage 1 ppm to 20 ppm, particularly 5 ppm to 15 ppm,
in coffee between 1 ppm and 40 ppm, particularly 15 ppm to 30 ppm,
in soy milk between 1 ppm and 20 ppm, particularly 5 ppm to 15 ppm,
in mayonnaise between 1 ppm and 10 ppm, particularly 1 ppm to 5 ppm,
in OTC syrup between 15 ppm and 40 ppm, particularly 25 ppm to 35 ppm,
in proteinaceous material between 5 ppm and 40 ppm, particularly 20 ppm to 35 ppm.

The situation is more complex with mixtures of the Compounds 1, 2 and 3, and the following figures should be taken as indicative only, as the skilled person will certainly be able to find other possible combinations and possibilities:

Beverage;

Compound 1:Compound 25 ppm:35 ppm up to 15 ppm:25 ppm,
Compound 1:Compound 3 ratio 5 ppm:35 ppm up to 15 ppm:1 ppm,

Coffee;

Compound 1:Compound 2 ratio 15 ppm:25 ppm up to 25 ppm:15 ppm,
Compound 1:Compound 3 ratio 15 ppm:15 ppm up to 25 ppm:5 ppm,

Soy milk;

Compound 1:Compound 2 ratio 5 ppm:15 ppm up to 15 ppm:5 ppm,
Compound 1:Compound 3 ratio 5 ppm:15 ppm up to 15 ppm:5 ppm,

Mayonnaise;

Compound 1:Compound 2 ratio 1 ppm:10 ppm up to 5 ppm:1 ppm,
Compound 1:Compound 3 ratio 1 ppm:15 ppm up to 5 ppm:5 ppm,

OTC syrup;

Compound 1:Compound 2 ratio 25 ppm:35 ppm up to 35 ppm:25 ppm,
Compound 1:Compound 3 ratio 25 ppm:25 ppm up to 35 ppm:15 ppm,

Proteinaceous material;

Compound 1:Compound 2 ratio 20 ppm:20 ppm up to 35 ppm:5 ppm,
Compound 1:Compound 3 ratio 20 ppm:20 ppm up to 35 ppm:5 ppm.

Compound 1 or masking composition may be used in such products with known flavor compounds, and also with all the known ancillary compounds used in flavoured products. They can be added and blended in the normal manner, like any other ingredient. In the case of the masking compositions, the individual components may be added separately, or pre-blended as a single additive.

The disclosure is further described with reference to the following examples, which describe certain embodiments, and which are not meant to be in any way limiting.

### EXAMPLES

The following compositions are made and tested blind. Using a blind bench-top tasting panel (consisting of 4 to 8 panelists), panelists were asked to record the sensory attribute differences between the control and example samples, specifically focusing on off-notes (reduction or differences).

The compounds are identified as follows:
Compound 1=1-(2-hydroxyphenyl)-3-(pyridin-4-yl)propane-1-one,
Compound 2=4-[2,2,3-trimethyl-cyclopentyl]-butanoic acid,
Compound 3=2-[(3-ethyl-4,8-dimethyl-2-oxo-2H-chromen-7-yl)oxy]propanoic acid.

All three compounds were used in the form of their hydrochloride salts.

### Example #1

### Full Sugar Energy Drink (Amp Energy™)

In this example, the drink used was Amp Energy™. This energy drink has the typical off-note characteristic of drinks based on caffeine, guarana and the like.

The proportions are as shown in Table 1.

TABLE 1

| | | Compound proportion | Drink proportion |
|---|---|---|---|
| Sample No. | Compound No. | (% by weight) | (% by weight) |
| 1 | — | — | 100 |
| 2 | 1 | 0.001 | to 100 |
| 3 | 2 | 0.001 | to 100 |
| 4 | 1/2 | 0.001/0.001 | to 100 |
| 5 | 3 | 0.0005 | to 100 |
| 6 | 1/3 | 0.001/0.0005 | to 100 |

Bench Taste Findings:

Panelists detected significantly less off-note in Sample 4 (0.001% of each of Compounds 1 and 2) over either Sample 3 (Compound 2 alone) or Sample 1 (control, no addition).

Panelists detected significantly less off-note in either Sample 2 or Sample 5 over the control. In Sample 6, there was detected significantly less off-note over the control.

### Example #2

### Full Sugar Energy Drink (Red Bull™)

The procedure of Example 1 was repeated using the proportions shown in Table 2:

TABLE 2

| | | Compound proportion | Drink proportion |
|---|---|---|---|
| Sample No. | Compound No. | (% by weight) | (% by weight) |
| 7 | — | — | 100 |
| 8 | 1 | 0.001 | to 100 |
| 9 | 2 | 0.001 | to 100 |
| 10 | 1/2 | 0.001/0.001 | to 100 |
| 11 | 3 | 0.0005 | to 100 |
| 12 | 1/3 | 0.001/0.0005 | to 100 |

Bench Taste Findings:

Panelists detected significantly less off-note in Sample 10 (0.001% of each of Compounds 1 and 2) over either Sample 8 (Compound 2 alone) or Sample 7 (control, no addition).

Panelists detected significantly less off-note in either Sample 8 or Sample 11 over the control. In Sample 12, there was detected significantly less off-note over the control.

### Example #3

### Diet Energy Drink

The drink used was Amp Energy™—Sugar Free. This drinks contains the artificial sweeteners sucralose and acesulfame-K, which are noted for having metallic off-tastes.

The procedure was carried out as described in Example 1, using the proportions shown in Table 3.

TABLE 3

| | | Compound proportion | Drink proportion |
|---|---|---|---|
| Sample No. | Compound No. | (% by weight) | (% by weight) |
| 13 | — | — | 100 |
| 14 | 1 | 0.001 | to 100 |
| 15 | 2 | 0.001 | to 100 |
| 16 | 1/2 | 0.001/0.001 | to 100 |
| 17 | 3 | 0.0005 | to 100 |
| 18 | 1/3 | 0.001/0.0005 | to 100 |

Bench Taste Findings:

Panelists preferred all the samples over the control. Sample 16 was preferred over Sample 15.

Panelists preferred Sample 18 to either Sample 14 or Sample 17.

### Example #4

### Diet Carbonated Soft Drink (CSD)

The drink used was Tab™, a saccharin-sweetened drink. Saccharin is known to contribute an off-taste.

The procedure was carried out as described in Example 1, using the proportions shown in Table 4.

TABLE 4

| | | Compound proportion | Drink proportion |
|---|---|---|---|
| Sample No. | Compound No. | (% by weight) | (% by weight) |
| 19 | — | — | 100 |
| 20 | 1 | 0.00025 | to 100 |
| 21 | 1 | 0.0005 | to 100 |
| 22 | 2 | 0.0005 | to 100 |
| 23 | 2 | 0.001 | to 100 |
| 24 | 3 | 0.0005 | to 100 |
| 25 | 3 | 0.001 | to 100 |
| 26 | 1/2 | 0.0005/0.0005 | to 100 |
| 27 | 1/3 | 0.0005/0.001 | to 100 |

Bench Taste Findings:

Panelists preferred Samples 21, 22, 24, 25, 26 and 27 over the control Sample 19.

### Example #5

### Diet Carbonated Soft Drink (CSD)

The drink used was Pepsi Max™, a drink sweetened with aspartame and acesulfame-K.

The procedure was carried out as described in Example 1, using the proportions shown in Table 5.

TABLE 5

| | | Compound proportion | Drink proportion |
|---|---|---|---|
| Sample No. | Compound No. | (% by weight) | (% by weight) |
| 28 | — | — | 100 |
| 29 | 1 | 0.001 | to 100 |
| 30 | 2 | 0.001 | to 100 |
| 31 | 1/2 | 0.001/0.001 | to 100 |
| 32 | 3 | 0.0005 | to 100 |
| 33 | 1/3 | 0.001/0.0005 | to 100 |

Bench Taste Findings:

The panelists preferred all the samples over the control. Panelists preferred Sample 31 over Sample 30, and Sample 33 over Sample 32.

### Example #6

### Diet Carbonated Soft Drink (CSD)

The drink was Zevia™, sweetened with Rebaudioside A.

The procedure was carried out as described in Example 1, using the proportions shown in Table 6.

TABLE 6

| | | Compound proportion | Drink proportion |
|---|---|---|---|
| Sample No. | Compound No. | (% by weight) | (% by weight) |
| 34 | — | — | 100 |
| 35 | 1 | 0.0005 | to 100 |
| 36 | 2 | 0.003 | to 100 |
| 37 | 1/2 | 0.0005/0.003 | to 100 |
| 38 | 3 | 0.003 | to 100 |
| 39 | 1/3 | 0.0005/0.0003 | to 100 |

Bench Taste Findings:

Panelists preferred Sample 37 over Samples 35, 36 and 38.

### Example #7

### Coffee (Unsweetened)

The procedure was carried out as described in Example 1, using the proportions shown in Table 7.

TABLE 7

| | | Compound proportion | Drink proportion |
|---|---|---|---|
| Sample No. | Compound No. | (% by weight) | (% by weight) |
| 40 | — | — | 100 |
| 41 | 1 | 0.002 | to 100 |
| 42 | 1 | 0.003 | to 100 |
| 43 | 2 | 0.002 | to 100 |
| 44 | 2 | 0.003 | to 100 |
| 45 | 3 | 0.001 | to 100 |
| 46 | 3 | 0.002 | to 100 |
| 47 | 1/2 | 0.002/0.003 | to 100 |
| 48 | 1/3 | 0.002/0.001 | to 100 |

Bench Taste Findings:
(i) The panelists preferred Samples 41, 44 and 45 over the control;
(ii) The panelists preferred Sample 47 over Sample 44;
(iii) The panelists preferred either Sample 41 or Sample 45.

### Example #8

### Plain Soymilk (Private Selection Organic Plain Soymilk—7 g Protein Per 8 oz Serving)

The procedure was carried out as described in Example 1, using the proportions shown in Table 8.

TABLE 8

| | | Compound proportion | Drink proportion |
|---|---|---|---|
| Sample No. | Compound No. | (% by weight) | (% by weight) |
| 49 | — | — | 100 |
| 50 | 1 | 0.001 | to 100 |
| 51 | 1 | 0.002 | to 100 |
| 52 | 2 | 0.001 | to 100 |
| 53 | 2 | 0.002 | to 100 |
| 54 | 3 | 0.0005 | to 100 |
| 55 | 3 | 0.001 | to 100 |
| 56 | 1/2 | 0.001/0.001 | to 100 |
| 57 | 1/3 | 0.001/0.001 | to 100 |

Bench Taste Findings:
(i) Panelists preferred Sample 50 over the control.
(ii) Panelists preferred Sample 52 over the control.
(iii) Panelists preferred Sample 54 over the control.
(iv) Panelists preferred the Sample 56 over Sample 52.
(v) Panelists preferred Sample 57 over Sample 50 or Sample 55 alone.

### Example #9

### Soy Yogurt (O'Soy Organic Soy Yogurt Strawberry Flavor—5 g Protein Per 4 oz Serving)

| Ingredient | % (by weight) |
|---|---|
| Soy Yogurt (O'Soy Organic Soy Yogurt | 100 |
| Strawberry Flavor) | |

| Ingredient | % (by weight) |
|---|---|
| Compound 1 | 0.001 |
| Soy Yogurt (O'Soy Organic Soy Yogurt | Balance to 100 |
| Strawberry Flavor) | |

| Ingredient | % (by weight) |
|---|---|
| Compound 1 | 0.002 |
| Soy Yogurt (O'Soy Organic Soy Yogurt | Balance to 100 |
| Strawberry Flavor) | |

Bench Taste Findings:

Panelists preferred 0.002% of Compound 1 over the control. The off-notes in this product are typically associated with soy protein.

### Example #10

### Soy Yogurt (O'Soy Organic Soy Yogurt Strawberry Flavor—5 g Protein Per 4 oz Serving)

| Ingredient | % (by weight) |
|---|---|
| Soy Yogurt (O'Soy Organic Soy Yogurt | 100 |
| Strawberry Flavor) | |

| Ingredient | % (by weight) |
|---|---|
| Compound 2 | 0.001 |
| Soy Yogurt (O'Soy Organic Soy Yogurt | Balance to 100 |
| Strawberry Flavor) | |

| Ingredient | % (by weight) |
|---|---|
| Compound 2 | 0.002 |
| Soy Yogurt (O'Soy Organic Soy Yogurt | Balance to 100 |
| Strawberry Flavor) | |

Bench Taste Findings:

Panelists preferred 0.002% of Compound 2 over the control. The off-notes in this product are typically associated with soy protein.

### Example #11

### Soy Yogurt (O'Soy Organic Soy Yogurt Strawberry Flavor—5 g Protein Per 4 oz Serving)

| Ingredient | % (by weight) |
|---|---|
| Soy Yogurt (O'Soy Organic Soy Yogurt | 100 |
| Strawberry Flavor) | |

| Ingredient | % (by weight) |
|---|---|
| Compound 3 | 0.0005 |
| Soy Yogurt (O'Soy Organic Soy Yogurt | Balance to 100 |
| Strawberry Flavor) | |

| Ingredient | % (by weight) |
|---|---|
| Compound 3 | 0.001 |
| Soy Yogurt (O'Soy Organic Soy Yogurt | Balance to 100 |
| Strawberry Flavor) | |

Bench Taste Findings:

Panelists preferred 0.001% of Compound 3 over the control. The off-notes in this product are typically associated with soy protein.

### Example #12

### Soy Yogurt (O'Soy Organic Soy Yogurt Strawberry Flavor—5 g Protein Per 4 oz Serving)

| Ingredient | % (by weight) |
|---|---|
| Soy Yogurt (O'Soy Organic Soy Yogurt | 100 |
| Strawberry Flavor) | |

| Ingredient | % (by weight) |
|---|---|
| Compound 1 | 0.002 |
| Soy Yogurt (O'Soy Organic Soy Yogurt | Balance to 100 |
| Strawberry Flavor) | |

| Ingredient | % (by weight) |
|---|---|
| Compound 3 | 0.001 |
| Soy Yogurt (O'Soy Organic Soy Yogurt | Balance to 100 |
| Strawberry Flavor) | |

| Ingredient | % (by weight) |
|---|---|
| Compound 1 | 0.002 |
| Compound 3 | 0.001 |
| Soy Yogurt (O'Soy Organic Soy Yogurt | Balance to 100 |
| Strawberry Flavor) | |

Bench Taste Findings:

Panelists preferred the blend of Compound 1 @ 0.002% and Compound 3 @ 0.001% over Compound 1 @ 0.002% or Compound 3 @ 0.001% alone. The off-notes in this product are typically associated with soy protein.

### Example #13

### Fortified Mayonnaise (Smart Balance Omega Plus Light Mayonnaise Dressing—Omega-3 (500 Mg Per 15 g Serving) and Plant Sterols (100 Mg Per 15 g Serving)

| Ingredient | % (by weight) |
|---|---|
| Fortified Mayonnaise (Smart Balance Omega | 100 |
| Plus Light Mayonnaise Dressing) | |

| Ingredient | % (by weight) |
|---|---|
| Compound 1 | 0.00025 |
| Fortified Mayonnaise (Smart Balance Omega | Balance to 100 |
| Plus Light Mayonnaise Dressing) | |

| Ingredient | % (by weight) |
|---|---|
| Compound 1 | 0.00025 |
| Fortified Mayonnaise (Smart Balance Omega | Balance to 100 |
| Plus Light Mayonnaise Dressing) | |

Bench Taste Findings:

Panelists preferred 0.00025% of Compound 1 over the control. The off-notes in this product are typically associated with omega-3 and plant sterols.

### Example #14

### Fortified Mayonnaise (Smart Balance Omega Plus Light Mayonnaise Dressing—Omega-3 (500 Mg Per 15 g Serving) and Plant Sterols (100 Mg Per 15 g Serving)

| Ingredient | % (by weight) |
|---|---|
| Fortified Mayonnaise (Smart Balance Omega | 100 |
| Plus Light Mayonnaise Dressing) | |

| Ingredient | % (by weight) |
|---|---|
| Compound 2 | 0.0005 |
| Fortified Mayonnaise (Smart Balance Omega | Balance to 100 |
| Plus Light Mayonnaise Dressing) | |

| Ingredient | % (by weight) |
|---|---|
| Compound 2 | 0.001 |
| Fortified Mayonnaise (Smart Balance Omega | Balance to 100 |
| Plus Light Mayonnaise Dressing) | |

Bench Taste Findings:

Panelists preferred 0.0005% of Compound 2 over the control. The off-notes in this product are typically associated with omega-3 and plant sterols.

### Example #15

### Fortified Mayonnaise (Smart Balance Omega Plus Light Mayonnaise Dressing—Omega-3 (500 Mg Per 15 g Serving) and Plant Sterols (100 Mg Per 15 g Serving)

| Ingredient | % (by weight) |
|---|---|
| Fortified Mayonnaise (Smart Balance Omega | 100 |
| Plus Light Mayonnaise Dressing) | |

| Ingredient | % (by weight) |
|---|---|
| Compound 3 | 0.0005 |
| Fortified Mayonnaise (Smart Balance Omega | Balance to 100 |
| Plus Light Mayonnaise Dressing) | |

| Ingredient | % (by weight) |
|---|---|
| Compound 3 | 0.001 |
| Fortified Mayonnaise (Smart Balance Omega | Balance to 100 |
| Plus Light Mayonnaise Dressing) | |

Bench Taste Findings:

Panelists preferred 0.001% of Compound 3 over the control. The off-notes in this product are typically associated with omega-3 and plant sterols.

### Example #16

### Fortified Mayonnaise (Smart Balance Omega Plus Light Mayonnaise Dressing—Omega-3 (500 Mg Per 15 g Serving) and Plant Sterols (100 Mg Per 15 g Serving)

| Ingredient | % (by weight) |
|---|---|
| Fortified Mayonnaise (Smart Balance Omega | 100 |
| Plus Light Mayonnaise Dressing) | |

| Ingredient | % (by weight) |
|---|---|
| Compound 1 | 0.00025 |
| Fortified Mayonnaise (Smart Balance Omega | Balance to 100 |
| Plus Light Mayonnaise Dressing) | |

| Ingredient | % (by weight) |
|---|---|
| Compound 2 | 0.0005 |
| Fortified Mayonnaise (Smart Balance Omega | Balance to 100 |
| Plus Light Mayonnaise Dressing) | |

| Ingredient | % (by weight) |
|---|---|
| Compound 1 | 0.00025 |
| Compound 2 | 0.0005 |
| Fortified Mayonnaise (Smart Balance Omega | Balance to 100 |
| Plus Light Mayonnaise Dressing) | |

Bench Taste Findings:

Panelists preferred the blend of Compound 1 @ 0.00025% and Compound 2 @ 0.0005% over Compound 1 @ 0.00025% and Compound 2 @ 0.0005% alone. The off-notes in this product are typically associated with omega-3 and plant sterols.

### Example #17

### Fortified Mayonnaise (Smart Balance Omega Plus Light Mayonnaise Dressing—Omega-3 (500 Mg Per 15 g Serving) and Plant Sterols (100 Mg Per 15 g Serving)

| Ingredient | % (by weight) |
|---|---|
| Fortified Mayonnaise (Smart Balance Omega | 100 |
| Plus Light Mayonnaise Dressing) | |

| Ingredient | % (by weight) |
|---|---|
| Compound 1 | 0.00025 |
| Fortified Mayonnaise (Smart Balance Omega | Balance to 100 |
| Plus Light Mayonnaise Dressing) | |

| Ingredient | % (by weight) |
|---|---|
| Compound 3 | 0.001 |
| Fortified Mayonnaise (Smart Balance Omega | Balance to 100 |
| Plus Light Mayonnaise Dressing) | |

| Ingredient | % (by weight) |
|---|---|
| Compound 1 | 0.00025 |
| Compound 3 | 0.001 |
| Fortified Mayonnaise (Smart Balance | Balance to 100 |
| Omega Plus Light Mayonnaise Dressing) | |

Bench Taste Findings:

Panelists preferred either Compound 1 @ 0.00025% OR Compound 3 @ 0.001% over the control. The blend of Compound 1 @ 0.00025% and Compound 3 @ 0.001% was preferred over the control but not necessarily over the individual ingredients (not enough tasters to determine statistics). The off-notes in this product are typically associated with omega-3 and plant sterols.

### Example #18

### Over-the-Counter (OTC) Syrup (Kroger Daytime Cold/Flu Relief (Acetaminophen 325 mg per 15 mL; Dextromethorphan HBr 10 mg per 15 mL; Phenylephrine HCl 5 mg per 15 mL))

| Ingredient | % (by weight) |
|---|---|
| OTC Syrup (Kroger Daytime Cold/Flu | 100 |
| Relief) | |

| Ingredient | % (by weight) |
|---|---|
| Compound 1 | 0.002 |
| OTC Syrup (Kroger Daytime Cold/Flu | Balance to 100 |
| Relief) | |

| Ingredient | % (by weight) |
|---|---|
| Compound 1 | 0.003 |
| OTC Syrup (Kroger Daytime Cold/Flu | Balance to 100 |
| Relief) | |

Bench Taste Findings:

Panelists preferred 0.003% of Compound 1 over the control. The off-notes in this product are typically associated with OTC syrups.

### Example #19

### Over-the-Counter (OTC) Syrup (Kroger Daytime Cold/Flu Relief (Acetaminophen 325 mg per 15 mL; Dextromethorphan HBr 10 mg per 15 mL; Phenylephrine HCl 5 mg per 15 mL))

| Ingredient | % (by weight) |
|---|---|
| OTC Syrup (Kroger Daytime Cold/Flu | 100 |
| Relief) | |

| Ingredient | % (by weight) |
|---|---|
| Compound 2 | 0.002 |
| OTC Syrup (Kroger Daytime Cold/Flu | Balance to 100 |
| Relief) | |

| Ingredient | % (by weight) |
|---|---|
| Compound 2 | 0.003 |
| OTC Syrup (Kroger Daytime Cold/Flu | Balance to 100 |
| Relief) | |

Bench Taste Findings:

Panelists preferred 0.003% of Compound 2 over the control. The off-notes in this product are typically associated with OTC syrups.

### Example #20

### Over-the-Counter (OTC) Syrup (Kroger Daytime Cold/Flu Relief (Acetaminophen 325 mg per 15 mL; Dextromethorphan HBr 10 mg per 15 mL; Phenylephrine HCl 5 mg per 15 mL))

| Ingredient | % (by weight) |
|---|---|
| OTC Syrup (Kroger Daytime Cold/Flu | 100 |
| Relief) | |

| Ingredient | % (by weight) |
|---|---|
| Compound 3 | 0.001 |
| OTC Syrup (Kroger Daytime Cold/Flu | Balance to 100 |
| Relief) | |

| Ingredient | % (by weight) |
|---|---|
| Compound 2 | 0.002 |
| OTC Syrup (Kroger Daytime Cold/Flu | Balance to 100 |
| Relief) | |

Bench Taste Findings:

Panelists preferred 0.002% of Compound 3 over the control. The off-notes in this product are typically associated with OTC syrups.

### Example #21

### Over-the-Counter (OTC) Syrup (Kroger Daytime Cold/Flu Relief (Acetaminophen 325 mg per 15 mL; Dextromethorphan HBr 10 mg per 15 mL; Phenylephrine HCl 5 mg per 15 mL))

| Ingredient | % (by weight) |
|---|---|
| OTC Syrup (Kroger Daytime Cold/Flu | 100 |
| Relief) | |

| Ingredient | % (by weight) |
|---|---|
| Compound 1 | 0.003 |
| OTC Syrup (Kroger Daytime Cold/Flu | Balance to 100 |
| Relief) | |

| Ingredient | % (by weight) |
|---|---|
| Compound 3 | 0.002 |
| OTC Syrup (Kroger Daytime Cold/Flu | Balance to 100 |
| Relief) | |

| Ingredient | % (by weight) |
|---|---|
| Compound 1 | 0.003 |
| Compound 3 | 0.002 |
| OTC Syrup (Kroger Daytime Cold/Flu | Balance to 100 |
| Relief) | |

Bench Taste Findings:

Panelists preferred the blend of Compound 1 @ 0.003% and Compound 3 @ 0.002% over Compound 1 @ 0.003% and Compound 3 @ 0.002% alone. The off-notes in this product are typically associated with OTC syrups.

### Example #22

### Over-the-Counter (OTC) Syrup (Kroger Daytime Cold/Flu Relief (Acetaminophen 325 mg per 15 mL; Dextromethorphan HBr 10 mg per 15 mL; Phenylephrine HCl 5 mg per 15 mL))

| Ingredient | % (by weight) |
|---|---|
| OTC Syrup (Kroger Daytime Cold/Flu | 100 |
| Relief) | |

| Ingredient | % (by weight) |
|---|---|
| Compound 1 | 0.003 |
| OTC Syrup (Kroger Daytime Cold/Flu | Balance to 100 |
| Relief) | |

| Ingredient | % (by weight) |
|---|---|
| Compound 2 | 0.003 |
| OTC Syrup (Kroger Daytime Cold/Flu | Balance to 100 |
| Relief) | |

| Ingredient | % (by weight) |
|---|---|
| Compound 1 | 0.003 |
| Compound 2 | 0.003 |
| OTC Syrup (Kroger Daytime Cold/Flu | Balance to 100 |
| Relief) | |

Bench Taste Findings:

Panelists preferred either Compound 1 @ 0.003% OR Compound 2 @ 0.003% over the control. The blend of Compound 1 @ 0.003% and Compound 2 @ 0.003% was preferred over the control but not necessarily over the individual ingredients (not enough tasters to determine statistics). The off-notes in this product are typically associated with OTC syrups.

### Example #23

### Proteinaceous Supplement (GNC Pro Performance 100% Whey Protein)

| Ingredient | % (by weight) |
|---|---|
| Proteinaceous Supplement (GNC Pro | 100 |
| Performance 100% Whey Protein) | |

| Ingredient | % (by weight) |
|---|---|
| Compound 1 | 0.00075 |
| Proteinaceous Supplement (GNC Pro | Balance to 100 |
| Performance 100% Whey Protein) | |

| Ingredient | % (by weight) |
|---|---|
| Compound 1 | 0.0015 |
| Proteinaceous Supplement (GNC Pro | Balance to 100 |
| Performance 100% Whey Protein) | |

| Ingredient | % (by weight) |
|---|---|
| Compound 1 | 0.003 |
| Proteinaceous Supplement (GNC Pro | Balance to 100 |
| Performance 100% Whey Protein) | |

Bench Taste Findings:

Panelists preferred Compound 1 @ 0.003% over the control. The off-notes in this product are typically associated with whey protein.

### Example #24

### Proteinaceous Supplement (GNC Pro Performance 100% Whey Protein)

| Ingredient | % (by weight) |
|---|---|
| Proteinaceous Supplement (GNC Pro | 100 |
| Performance 100% Whey Protein) | |

| Ingredient | % (by weight) |
|---|---|
| Compound 2 | 0.00075 |
| Proteinaceous Supplement (GNC Pro | Balance to 100 |
| Performance 100% Whey Protein) | |

| Ingredient | % (by weight) |
|---|---|
| Compound 2 | 0.0015 |
| Proteinaceous Supplement (GNC Pro | Balance to 100 |
| Performance 100% Whey Protein) | |

| Ingredient | % (by weight) |
|---|---|
| Compound 2 | 0.0015 |
| Proteinaceous Supplement (GNC Pro | Balance to 100 |
| Performance 100% Whey Protein) | |

Bench Taste Findings:

Panelists preferred Compound 2 @ 0.0015% over the control. The off-notes in this product are typically associated with whey protein.

### Example #25

### Proteinaceous Supplement (GNC Pro Performance 100% Whey Protein)

| Ingredient | % (by weight) |
|---|---|
| Proteinaceous Supplement (GNC Pro | 100 |
| Performance 100% Whey Protein) | |

| Ingredient | % (by weight) |
|---|---|
| Compound 3 | 0.00075 |
| Proteinaceous Supplement (GNC Pro | Balance to 100 |
| Performance 100% Whey Protein) | |

| Ingredient | % (by weight) |
|---|---|
| Compound 3 | 0.0015 |
| Proteinaceous Supplement (GNC Pro | Balance to 100 |
| Performance 100% Whey Protein) | |

| Ingredient | % (by weight) |
|---|---|
| Compound 3 | 0.0015 |
| Proteinaceous Supplement (GNC Pro | Balance to 100 |
| Performance 100% Whey Protein) | |

Bench Taste Findings:

Panelists preferred Compound 3 @ 0.0015% over the control. The off-notes in this product are typically associated with whey protein.

### Example #26

### Proteinaceous Supplement (GNC Pro Performance 100% Whey Protein)

| Ingredient | % (by weight) |
|---|---|
| Proteinaceous Supplement (GNC Pro | 100 |
| Performance 100% Whey Protein) | |

| Ingredient | % (by weight) |
|---|---|
| Compound 1 | 0.003 |
| Proteinaceous Supplement (GNC Pro | Balance to 100 |
| Performance 100% Whey Protein) | |

| Ingredient | % (by weight) |
|---|---|
| Compound 2 | 0.0015 |
| Proteinaceous Supplement (GNC Pro | Balance to 100 |
| Performance 100% Whey Protein) | |

| Ingredient | % (by weight) |
|---|---|
| Compound 1 | 0.003 |
| Compound 2 | 0.0015 |
| Proteinaceous Supplement (GNC Pro | Balance to 100 |
| Performance 100% Whey Protein) | |

Bench Taste Findings:

Panelists preferred the blend of Compound 1 @ 0.003% and Compound 2 @ 0.0015% over Compound 2 @ 0.0015% alone. The off-notes in this product are typically associated with whey protein.

### Example #27

### Proteinaceous Supplement (GNC Pro Performance 100% Whey Protein)

| Ingredient | % (by weight) |
|---|---|
| Proteinaceous Supplement (GNC Pro | 100 |
| Performance 100% Whey Protein) | |

| Ingredient | % (by weight) |
|---|---|
| Compound 1 | 0.003 |
| Proteinaceous Supplement (GNC Pro | Balance to 100 |
| Performance 100% Whey Protein) | |

| Ingredient | % (by weight) |
|---|---|
| Compound 3 | 0.0015 |
| Proteinaceous Supplement (GNC Pro | Balance to 100 |
| Performance 100% Whey Protein) | |

| Ingredient | % (by weight) |
|---|---|
| Compound 1 | 0.003 |
| Compound 3 | 0.003 |
| Proteinaceous Supplement (GNC Pro | Balance to 100 |
| Performance 100% Whey Protein) | |

Bench Taste Findings:

Panelists preferred the blend of Compound 1 @ 0.003% and Compound 3 @ 0.0015% over Compound 1 @ 0.003% and Compound 3 @ 0.0015% alone. The off-notes in this product are typically associated with whey protein.

### Example #28

### Cocoa (Dilute 10% in Water to Consume) (Kroger Cocoa Unsweetened)

| Ingredient | % (by weight) |
|---|---|
| Cocoa (dilute 10% in water) (Kroger Cocoa | 100 |
| Unsweetened) | |

| Ingredient | % (by weight) |
|---|---|
| Compound 1 | 0.002 |
| Cocoa (dilute 10% in water) (Kroger Cocoa | Balance to 100 |
| Unsweetened) | |

| Ingredient | % (by weight) |
|---|---|
| Compound 1 | 0.003 |
| Cocoa (dilute 10% in water) (Kroger Cocoa | Balance to 100 |
| Unsweetened) | |

Bench Taste Findings:

Panelists preferred Compound 1 @ 0.002% over the control. The off-notes in this product are typically associated with cocoa (unsweetened).

### Example #29

### Cocoa (Dilute 10% in Water to Consume) (Kroger Cocoa Unsweetened)

| Ingredient | % (by weight) |
|---|---|
| Cocoa (dilute 10% in water) (Kroger Cocoa | 100 |
| Unsweetened) | |

| Ingredient | % (by weight) |
|---|---|
| Compound 3 | 0.001 |
| Cocoa (dilute 10% in water) (Kroger Cocoa | Balance to 100 |
| Unsweetened) | |

| Ingredient | % (by weight) |
|---|---|
| Compound 3 | 0.002 |
| Cocoa (dilute 10% in water) (Kroger Cocoa | Balance to 100 |
| Unsweetened) | |

Bench Taste Findings:

Panelists preferred Compound 3 @ 0.001% over the control. The off-notes in this product are typically associated with cocoa (unsweetened).

### Example #30

### Cocoa (Dilute 10% in Water to Consume) (Kroger Cocoa Unsweetened)

| Ingredient | % (by weight) |
|---|---|
| Cocoa (dilute 10% in water) (Kroger Cocoa | 100 |
| Unsweetened) | |

| Ingredient | % (by weight) |
|---|---|
| Compound 2 | 0.001 |
| Cocoa (dilute 10% in water) (Kroger Cocoa | Balance to 100 |
| Unsweetened) | |

| Ingredient | % (by weight) |
|---|---|
| Compound 3 | 0.002 |
| Cocoa (dilute 10% in water) (Kroger Cocoa | Balance to 100 |
| Unsweetened) | |

Bench Taste Findings:

Panelists preferred Compound 2 @ 0.002% over the control. The off-notes in this product are typically associated with cocoa (unsweetened).

### Example #31

### Cocoa (Dilute 10% in Water to Consume) (Kroger Cocoa Unsweetened)

| Ingredient | % (by weight) |
|---|---|
| Cocoa (dilute 10% in water) (Kroger Cocoa | 100 |
| Unsweetened) | |

| Ingredient | % (by weight) |
|---|---|
| Compound 1 | 0.002 |
| Cocoa (dilute 10% in water) (Kroger Cocoa | Balance to 100 |
| Unsweetened) | |

| Ingredient | % (by weight) |
|---|---|
| Compound 2 | 0.002 |
| Cocoa (dilute 10% in water) (Kroger Cocoa | Balance to 100 |
| Unsweetened) | |

| Ingredient | % (by weight) |
|---|---|
| Compound 1 | 0.002 |
| Compound 2 | 0.002 |
| Cocoa (dilute 10% in water) (Kroger Cocoa | Balance to 100 |
| Unsweetened) | |

Bench Taste Findings:

Panelists preferred either Compound 1 @ 0.002% OR Compound 2 @ 0.002% over the control. The blend of Compound 1 @ 0.003% and Compound 2 @ 0.003% was NOT preferred over the control. The off-notes in this product are typically associated with cocoa (unsweetened).

### Example #32

### Cocoa (Dilute 10% in Water to Consume) (Kroger Cocoa Unsweetened)

| Ingredient | % (by weight) |
|---|---|
| Cocoa (dilute 10% in water) (Kroger Cocoa | 100 |
| Unsweetened) | |

| Ingredient | % (by weight) |
|---|---|
| Compound 1 | 0.002 |
| Cocoa (dilute 10% in water) (Kroger Cocoa | Balance to 100 |
| Unsweetened) | |

| Ingredient | % (by weight) |
|---|---|
| Compound 3 | 0.001 |
| Cocoa (dilute 10% in water) (Kroger Cocoa | Balance to 100 |
| Unsweetened) | |

| Ingredient | % (by weight) |
|---|---|
| Compound 1 | 0.002 |
| Compound 3 | 0.001 |
| Cocoa (dilute 10% in water) (Kroger Cocoa | Balance to 100 |
| Unsweetened) | |

Bench Taste Findings:

Panelists preferred either Compound 1 @ 0.002% OR Compound 3 @ 0.001% over the control. The blend of Compound 1 @ 0.002% and Compound 3 @ 0.001% was NOT preferred over the control. The off-notes in this product are typically associated with cocoa (unsweetened).

Although the embodiments have been described in detail through the above description and the preceding examples, these examples are for the purpose of illustration only and it is understood that variations and modifications can be made by one skilled in the art without departing from the scope of the disclosure. It should be understood that the embodiments described above are not only in the alternative, but can be combined.

1. A method of masking an off-note in an orally-receivable or -ingestible composition, comprising the addition thereto of an off-note masking quantity of 1-(2-hydroxyphenyl)-3-(pyridin-4-yl)propane-1-one or a salt thereof.

2. The method according to claim 1, in which there is additionally added to the composition a compound selected from the group consisting of 4-[2,2,3-trimethyl-cyclopentyl]-butanoic acid, 2-[(3-ethyl-4,8-dimethyl-2-oxo-2H-chromen-7-yl)oxy]propanoic acid, and salts thereof.

3. An off-note-masked orally-receivable or -ingestible composition comprising an off-note masking quantity of 1-(2-hydroxyphenyl)-3-(pyridin-4-yl)propane-1-one or a salt thereof.

4. An off-note-masked orally-receivable or -ingestible composition comprising an off-note masking quantity of a masking composition comprising 1-(2-hydroxyphenyl)-3-(pyridin-4-yl)propane-1-one and at least one of 4-[2,2,3-trimethyl-cyclopentyl]-butanoic acid or 2-[(3-ethyl-4,8-dimethyl-2-oxo-2H-chromen-7-yl)oxy]propanoic acid or salts thereof.

5. An off-note-masking composition consisting of 1-(2-hydroxyphenyl)-3-(pyridin-4-yl)propane-1-one and at least one of 4-[2,2,3-trimethyl-cyclopentyl]-butanoic acid or 2-[(3-ethyl-4,8-dimethyl-2-oxo-2H-chromen-7-yl)oxy]propanoic acid or salts thereof.

6. The off-note-masking composition according to claim 5, consisting of 1-(2-hydroxyphenyl)-3-(pyridin-4-yl)propane-1-one and 4-[2,2,3-trimethyl-cyclopentyl]-butanoic acid, or salts thereof.

7. The off-note-masking composition according to claim 5, consisting of 1-(2-hydroxyphenyl)-3-(pyridin-4-yl)propane-1-one and 2-[(3-ethyl-4,8-dimethyl-2-oxo-2H-chromen-7-yl)oxy]propanoic acid, or salts thereof.

8. The off-note-masking composition according to claim 5, consisting of 1-(2-hydroxyphenyl)-3-(pyridin-4-yl)propane-1-one, 4-[2,2,3-trimethyl-cyclopentyl]-butanoic acid and 2-[(3-ethyl-4,8-dimethyl-2-oxo-2H-chromen-7-yl)oxy]propanoic acid, or salts thereof.
